(19) United States
(12) Patent Application Publication
(10) Pub. No.: US 2021/0228391 A1
(43) Pub. Date: Jul. 29, 2021

(54) WRIST BRACE

(71) Applicant: Dynacore Pty Ltd, St Kilda (AU)

(72) Inventors: Nicholas Criticos, St. Kilda (AU); Benjamin Cunningham, St. Kilda (AU)

(73) Assignee: Dynacore Pty Ltd, St Kilda (AU)

(21) Appl. No.: 17/154,035

(22) Filed: Jan. 21, 2021

(30) Foreign Application Priority Data
Jan. 24, 2020 (AU) 2020900198
Sep. 30, 2020 (AU) 2020903539

(51) Int. Cl. A61F 5/01

(52) U.S. Cl. CPC A61F 5/0118

(57) ABSTRACT

Disclosed is a brace for treating an injury of the distal radio-ulna joint of an individual. The brace may include a semi-rigid support member having a concave surface configured to extend longitudinally over a distal radius of the individual adjacent the individual's distal radio-ulna joint, and at least one retaining arm configured to be wound about the individual's arm to engage with an outer surface of the support member. The at least one retaining arm is formed from a resilient material such that tightening of the retaining arm about the individual's arm applies pressure to the distal radio-ulnar joint to alleviate pain and discomfort experienced therein.

### FIELD OF INVENTION

The present invention relates generally to an orthopaedic wrist brace, and in particular, to an orthopaedic wrist brace for the treatment of a distal radio-ulnar joint (DRUJ) injury.

### BACKGROUND OF THE INVENTION

Wrist injuries are a common type of injury experienced by people of all ages. Most wrist injuries are a direct result of an individual falling or tripping over and putting their hands down to break the fall. In such instances, significant forces are experiences at the individual's wrist which can cause injury at the DRUJ.

The distal radio-ulna joint (DRUJ) is the distal (far) joint between the radius and ulna bones of the forearm. These bones are linked just below the elbow and just above the wrist and do not meet, but are linked by a long stout ligament. The joints at the elbow (proximal radio-ulna joint) and at the wrist (DRUJ) allow the radius to rotate around the ulna. This enables the forearm to undergo pronation movement (turning the hand down to the floor) and supination movement (turning the hand up to the ceiling). The distal ulna bone also gives support to the wrist bones and at the far end of the ulna there is a ligament complex called the triangular fibrocartilaginous complex (TFCC) that stabilises the radius to the ulna and helps support the wrist.

The main problems individuals experience at the DRUJ are associated with instability of the joint, typically following an injury. Wrist fractures are often associated with tears of the TFCC or a fracture (break) of the ulna styloid to which the TFCC is attached. In most cases such fractures heal well with either no instability or mild instability with minimal or no symptoms. However, in some patients, they may experience aching over the ulnar (little finger) side of the wrist together with some stiffness of the joint, particularly in relation to supination movement (turning the palm up). Wrist fractures and soft tissue injuries can occur at all ages. Instability of the DRUJ generally results due to damage being sustained to the ligamentous complex that secures the radius to the ulna. When weakened this allows for excess movement at the DRUJ which may be both uncomfortable and cause stiffness.

A variety of treatment systems have been proposed to address pain and discomfort due to instability of the DRUJ. Many of these system involve the use of painkillers and anti-inflammatory analgesics that can be applied topically or orally to address the pain. A variety of splints and braces have also been proposed that apply pressure about the DRUJ, forearm, radiocarpal joint, metacarpals and around the base of the thumb in an attempt to stabilise the DRUJ. Many of these braces and splints are cumbersome to wear and are general purpose in design and not specific to DRUJ injuries. As such, most splints are largely ineffective and are mare of soft materials that merely compress the region rather than provide any support to the DRUJ and as such, provide only varying degrees of success.

Thus, there is a need to provide a brace for addressing issues associated with the DRUJ that provides support to the DRUJ to address and improve the underlying issues of the injury, rather than merely the symptoms of the injury.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### STATEMENT OF INVENTION

The invention according to one or more aspects is as defined in the independent claims. Some optional and/or preferred features of the invention are defined in the dependent claims.

Accordingly, in one aspect of the invention there is provided a brace for treating an injury of the distal radio-ulna joint of an individual, comprising:
a semi-rigid support member having a concave surface configured to extend longitudinally over a radius of the individual adjacent the individual's distal radio-ulna joint;
at least one retaining arm to be wound about the individual's arm to engage with an outer surface of the support member;
wherein the retaining arm is formed from a resilient material such that tightening of the retaining arm about the individual's arm applies pressure to the distal radio-ulnar joint to alleviate pain and discomfort experienced therein.

In one embodiment, the semi-rigid support member has a semi-cylindrical configuration that defines the concave surface so as to be fitted over and along the individual's radius, adjacent the distal radio-ulnar joint. The concave surface of the support member may be coated with a resilient layer to act as a cushion between the support member and the individual's skin. The support member may be flexible about a longitudinal axis but rigid about a radial axis.

The support member may cup the radius when worn.

In one embodiment one retaining arm is extendable from a longitudinal edge of the support member and is attachable by way of a hook and loop fastener to the outer surface of the support member.

The at least one retaining arm may be formed from a neoprene material.

The retaining arm and the support member have the same width.

In one embodiment, the support member is mounted over a pad member which is configured to be positioned between the support member and the individual's skin when in use.

In this embodiment, two retaining arms may be provided to extend angularly from opposing ends of the pad member. The two retaining arms may form a front retaining arm and a rear retaining arm which extend about a front region and a rear region of the support member to secure the brace in position on the arm of the individual.

The two retaining arms may be positioned so as to provide a right-handed version of the brace and a left-handed version of the brace for use on either a right or eft hand of the individual.

### BRIEF DESCRIPTION OF THE DRAWINGS

The invention may be better understood from the following non-limiting description of preferred embodiments, in which:

FIG. 1 is a perspective view of a brace in accordance with an embodiment of the present invention;

FIG. 2 is an end view of the brace of FIG. 1;

FIG. 3 is a side view of the brace of FIG. 1 in use;

FIG. 4 is a rear perspective view of the brace of FIG. 1 in;

FIG. 5 a rear perspective view of an alternative embodiment of the brace in accordance with the present invention;

FIG. 6 is a top view of a retaining arm for use in forming the brace in accordance with an embodiment of the present invention;

FIG. 7 is a perspective view of the retaining arm of FIG. 6;

FIG. 8 is an exploded view of an alternate embodiment of a brace in accordance with an embodiment of the present invention;

FIGS. 9 and 10 are left and right handed versions of the brace of the embodiment depicted in FIG. 8; and

FIG. 11 is a depiction of the brace of FIG. 8 positioned for fitting about a wrist of a patient.

### DETAILED DESCRIPTION OF THE DRAWINGS

Preferred features of the present invention will now be described with particular reference to the accompanying drawings. However, it is to be understood that the features illustrated in and described with reference to the drawings are not to be construed as limiting on the scope of the invention.

The present invention will be described below in relation to its application for use in treating an injury to the DRUJ, as may result from a fall or the like. However, it will be appreciated that the present invention could be used to treat other types of wrist injuries or wrist instabilities.

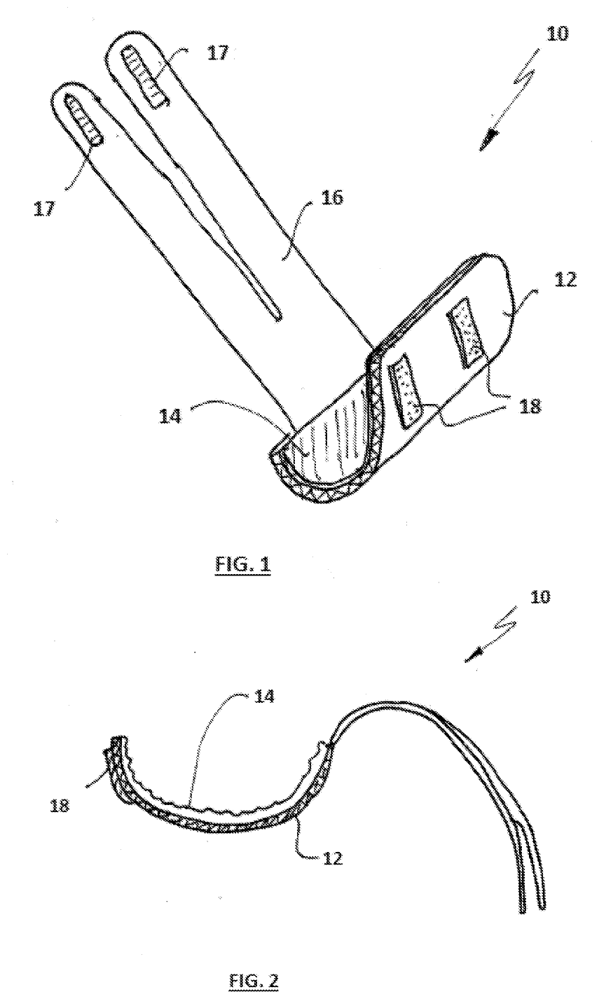

Referring to FIGS. 1 and 2, a wrist brace 10 is depicted for attachment about a wrist region of an individual suffering from pain or discomfort following an injury to the DRUJ. The brace 10 comprises a support portion 12 in the form of a semi-rigid, semi-cylindrical body having a concave inner surface that is configured to fit about a portion of the patient's wrist, such that the degree of curvature of the concave inner surface substantially conforms to the lateral shape of the patient's distal radius when worn. The support portion 12 is positioned on the patient's wrist so that is substantially cups the patient's distal radius.

The support portion 12 is made from a semi rigid material, such as a carbon fibre composite or plastic that has a degree of stiffness that substantially retains its shape, but which is able to flex when worn to maximise comfort to the patient. The inner concave surface of the support portion 12, which is in contact with the patient's skin, comprises a foam layer 14 so as to compress and conform to the patient's wrist/lower arm when worn, to maximise patient comfort. The foam layer 14 has a degree of resilience to function as a cushion between the patient's skin and the support portion such that the support portion 12 is able to be tightly secured to the patient's wrist without causing discomfort to the patient.

A retaining arm 16 extends from one edge of the support portion 12 to provide a means by which the brace 10 is secured to the patient. The retaining arm 16 is formed from a flexible fabric, such as neoprene, however any other suitable resiliently flexible material can be used for this purpose. As the retaining arm 16 is flexible, it can be stretched around the patient's arm to attach to the outer surface of the support portion 12 by way of a hook and loop fastener system, such as Velcro®. In this regard, the inside end of the retaining arm 16 may have one or more dedicated loop pads 17 provided on a surface thereof which engage with a respective hook portion pad 18 provided on the outer surface of the support portion 12. It will be appreciated that the inner surface of the retaining arm 16 may be configured with a material having loops that engage with the hook pads 18, thereby obviating the need to attach loop pads 17 to the retaining arm 16. Alternatively, other fastening means, such as press studs, buckles and the like, may be employed to secure the retaining arm in position.

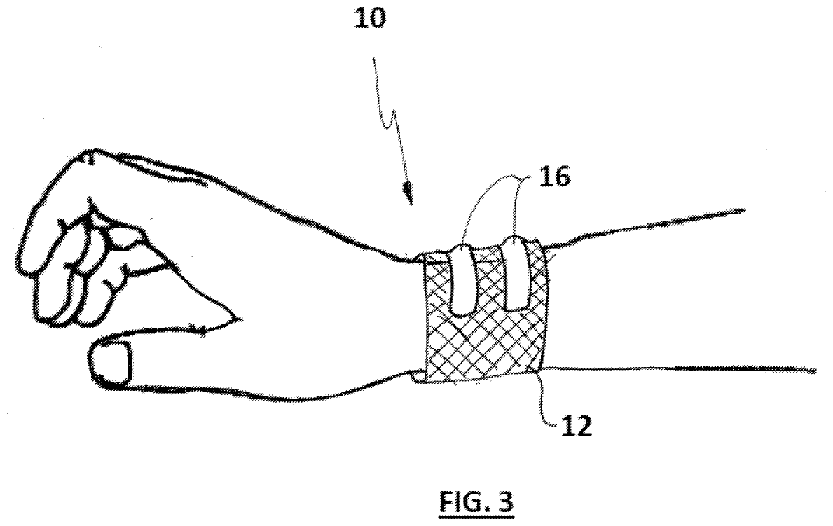

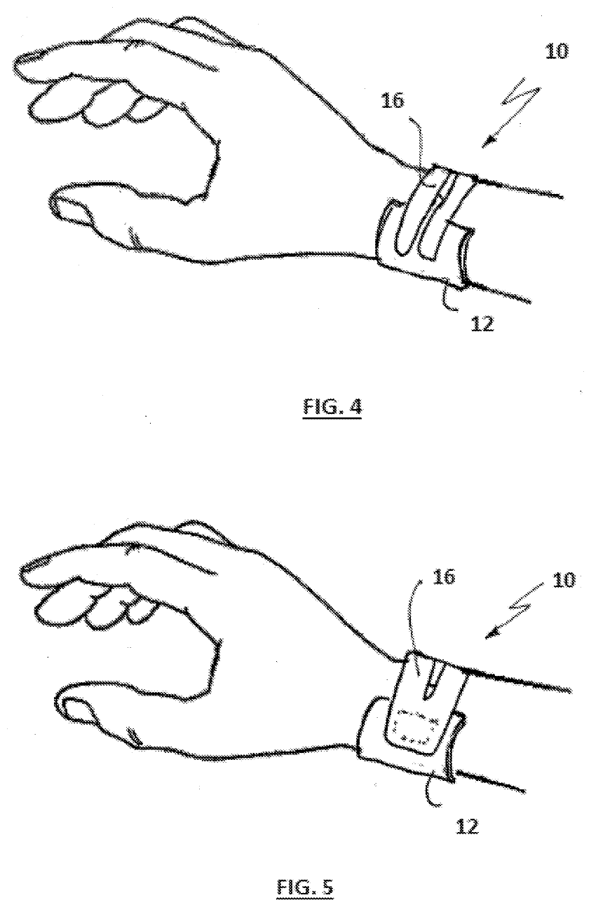

In the embodiment as shown in FIGS. 1-4, the retaining arm 16 has been split to comprise two portions for securing in position about the patient's arm, whilst FIG. 5 depicts a retaining arm 16 as a single band having a cut formed therein. The split portions and the single band with the cut formed therein are provided to accommodate the patient's ulnar styloid process. Thus, when attached about the wrist region of the patient, the split portions and the cut in the band enable the bony prominence on the ulnar styloid process to be free and visible, with respect to the remainder of the patient's wrist, with the attachment of the retaining arm 16 formed at the rigid radial portion.

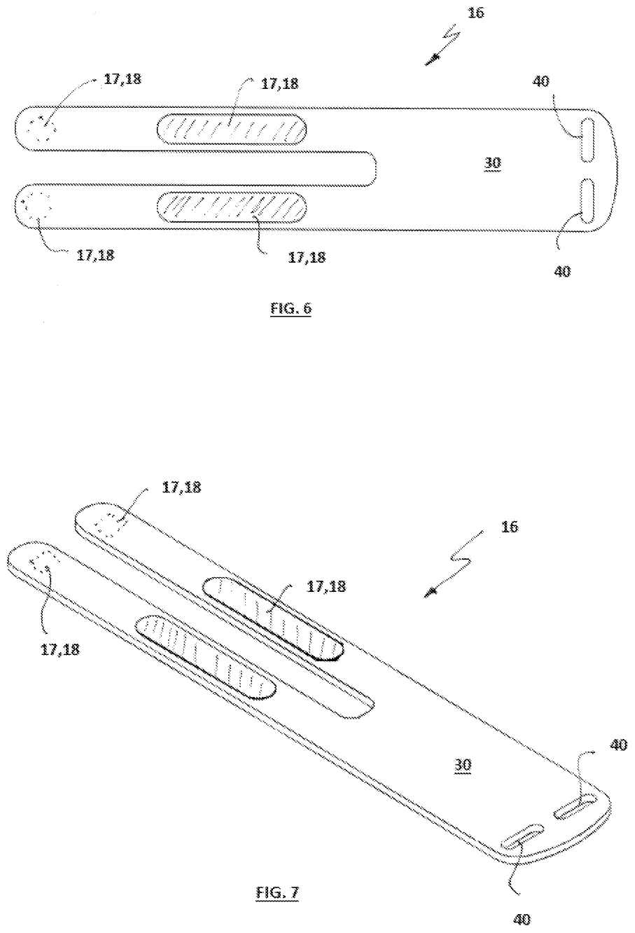

FIGS. 6 and 7 depict an alternative embodiment for the retaining arm 16 that employs split portions to accommodate the patient's ulnar styloid process in the same manner as FIGS. 1-4. However, in this embodiment, the retaining arm 16 is configured to have the support portion 12 mounted to an underside of the surface thereof adjacent the distal end of the retaining arm 16, where two slots 40 are formed therein. In this arrangement, when the brace is attached about the patient's wrist, the split portions of the retaining aim pass through a slot 40 to enable the split portions to be folded back and tightened about the patient's wrist. Velcro 17, 18 is used to engage the ends of the split portions with the body of the retaining arm 16.

As depicted more clearly in FIGS. 4 and 5, to wear the wrist brace 10, the support portion 12 is first fitted about the lateral side of the patient's radius, on the thumb side of the arm when the patient's hand is in a palm downwards position. As the support portion 12 is relatively rigid, it functions to cup the patient's radius in this position to enable the retaining arm 16 to pass around the patient's arm to reattach to the outer surface of the support portion 12 as shown. The retaining arm 16 can then be readjusted or tightened to ensure that the support portion is retained in position as the patient goes about their normal activities.

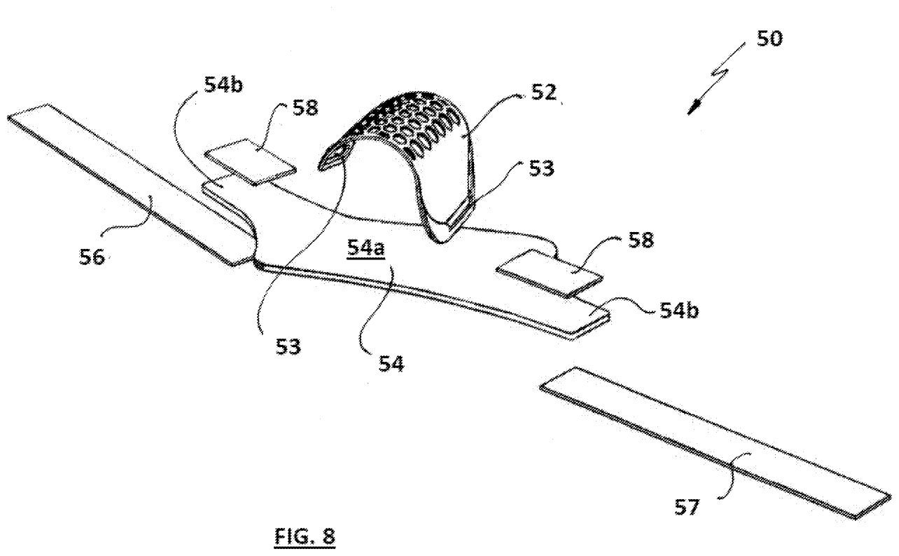

An alternative embodiment of a wrist brace in accordance with the present invention is depicted as reference numeral 50 in FIGS. 8-11.

In this embodiment, the wrist brace 50 functions to achieve the results in the same manner as described above. In the alternative embodiment as depicted, the wrist brace 50 comprises two opposing arm members 56 that extend from opposing sides of the brace member to secure the wrist brace 50 in position on the patient's arm and to apply additional support to the patient.

Referring to FIG. 8, the wrist brace 50 is depicted in exploded form. A support portion 52, which functions in the same manner as previously described support portion 12, is provided in a central region of the brace 50. The support portion 52 has a curved or substantial U-shape configuration and is substantially rigid in form so as to cup and support the patient's distal radius when worn. In a preferred embodiment the support portion 52 is made from a plastic material, such as Nylon, silicone, plastic or rubber which may be either 3D printed or injection moulded to form the desired shape. The walls of the support portion 52 may be between 1-5 mm in thickness and the body of the support portion 52 may have a plurality of holes, moulded or otherwise formed, through the walls thereof to provide a degree of flexibility to the support portion. A slot 53 is also formed at each end of the support portion 52 to facilitate attachment of the brace 50 to the patient, in a manner as will be described in more detail below.

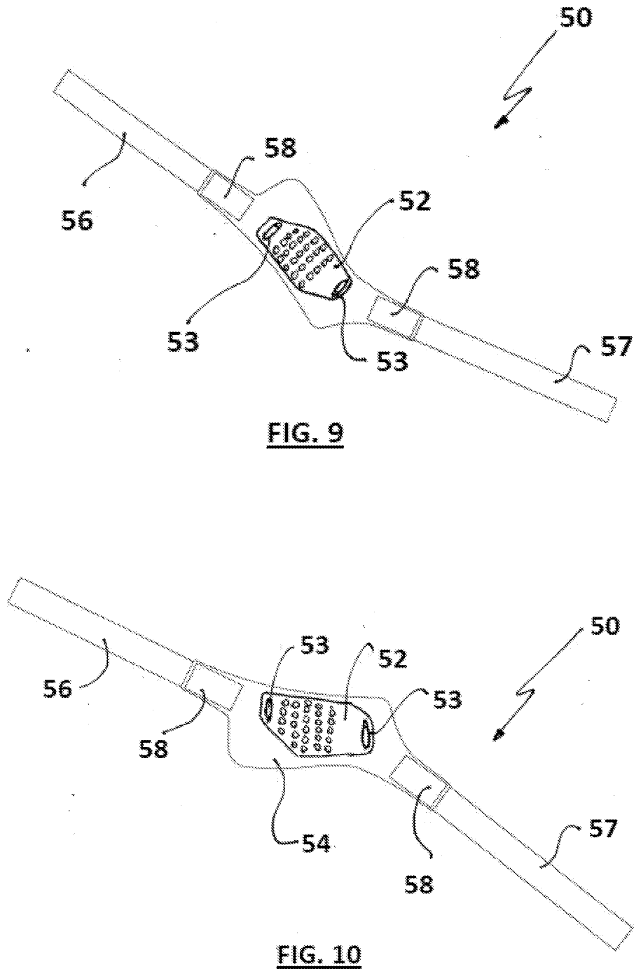

A pad member 54 is provided to carry the support member 52 thereon. The pad member 54 is made from a soft and stretchable material, such as Neoprene®, which may be perforated to provide a degree of ventilation for the wearer of the wrist brace 40. As can be more readily appreciated from FIGS. 9 and 10, the pad member 54 has a substantially rectangular main body portion 54a which is angularly offset from a pair of tab members 54b that extending angularly from opposing corners of the main body portion 54a, as shown.

The main body portion 54a is configured to be attached to an inside surface of the support member 52 by way of an adhesive, such as a flexible adhesive or glue.

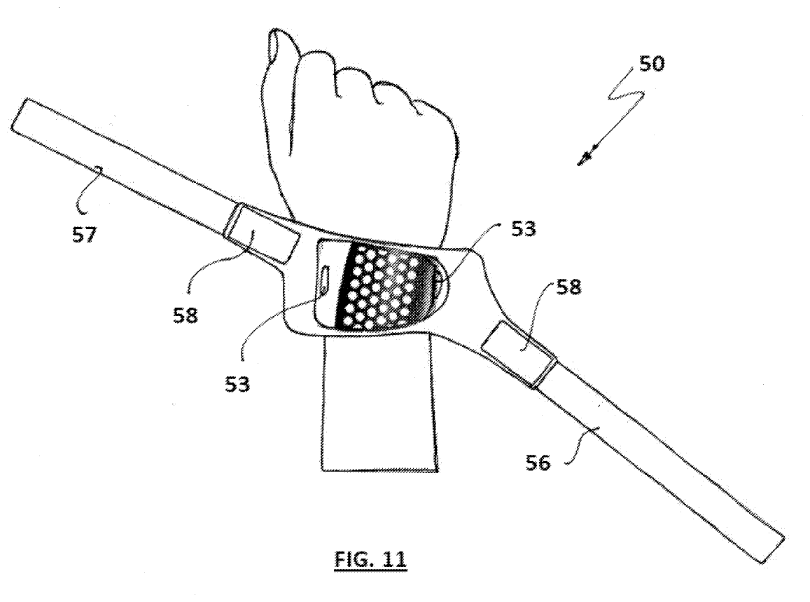

Alternatively, the main body portion 54a may be attached to an inside surface of the support member 52 by way ultrasonic wilding or other mechanical means. When the main body support member 52, the longitudinal sides of the main body portion 54a extend substantially parallel with the sides of the support member 52, as is shown in FIG. 11. In this regard, the underside of the main body portion 54a will be in contact with the skin of the patient when the brace 50 is positioned for use. In this arrangement, the main body portion 54a provides a degree of padding for the wearer and further padding may be added to the underside of the main body portion 54a to further maximise comfort for the patient. The underside of the main body portion 54a may also be surface treated to minimise slipping or movement of the wrist brace on the patient's skin.

The support portion 52 is secured to the main body portion 54a in a manner that retains the slots 53 of the support portion exposed for use. The upper surface of the tab member 54b are provided with engagement pads 58, such as strips of hook or loop material, such as Velcro. A pair of band members 56, 57 extend from the ends of each tab member 54b to facilitate attachment to the wrist of the patient. The band members 56, 57 are stitched or otherwise attached to the underside of the tab members 54b and may be formed from a loop material to enable attachment of the band members 56, 57 to the corresponding hook or loop material of the engagement pads 58.

As is depicted more clearly in FIGS. 9 and 10, the orientation of the wrist brace 50 is slightly different for left and right handed applications. FIG. 9 depicts a left handed wrist brace 50 whilst FIG. 10 depicts a righted handed wrist brace 50. Such a different orientation for use on different arms of the patient is required to ensure that in both situations there is separation of the band members 56, 57 over the ulnar, as well as counter rotation/lift of the carpus/wrist due to the action of the band members 56, 57. The different orientations of the wrist brace are achieved by rotating the orientation of the pad member 54, which varies the angles at which the band members 56, 57 extend from the pad member 54. In this regard, the pad member 54 is made to have a single orientation which can be rotated for use in both a right-handed and a left-handed application.

FIG. 11 depicts a right handed wrist brace 50 in use. To attach the wrist brace 50, the wrist brace is firstly orientated at right angles to the patient's arm, in the manner as shown. The support portion 52 is first fitted about the lateral side of the patient's radius, on the thumb side of the arm when the patient's hand is in a palm downwards position as shown. As the support portion 52 is relatively rigid, the support portion 52 functions to cup the patient's radius in this position.

In the fitting positon as shown in FIG. 11, the front band member 57 extends in front of the wrist at an angle as shown. The front band member 57 is then brought under the patient's wrist to extend under the wrist and be looped through the slot 53 located on the opposing side of the support portion 52. The end of the front band member 57 is then returned to engage with the engagement pad 58 located on the tab adjacent the base of the front band member 57. The front band member 57 can then be tightened as required to provide the desired degree of support. In this regard, the front band member 57 functions to lift up the carpus/wrist and to provide support to the pisiform/triquetrum bone.

The rear band member 56 is also brought under the patients arm so as to loop through the slot 53 located on the opposing side of the support member 52. The end of the rear band member 56 is then returned to engage with the engagement pad 58 located on the tab adjacent the base of the rear band member 56. Similarly, the rear band member 56 can be tightened so as to provide support behind the patient's ulnar styloid process. In use, the rear band member 56 applies a force to the distal ulnar to move volarly to provide greater ulnar-carpal alignment and DRUJ stabilisation. Due to the provision of the two band members 56, 57, the band members each separate around the patient's ulnar styloid process. Once positioned and correctly adjusted, the support portion is retained in position as the patient goes about their normal activities. In the left and right handed versions of the wrist brace 50, the brace 50 may be provided in a variety of different sizes to accommodate a variety of different sized patients.

It will be appreciated that the wrist brace 10, 50 is able to be unobtrusively worn by the patient and may be worn under a long sleeve shirt or similar clothing worn by the patient to conceal the brace 10. Preferably the wrist brace 10, 50 has a constant width across its entire length such that the width of the retaining arm and of the support portion are substantially the same.

In use, the support portion 12, 52 grips the patient's distal radius and provides a support point about which the wrist brace is wound. The retaining arm 16 or band member 56, 57 supply a force about the patient's lower arm that functions to provide a degree of stability to the DRUJ that minimises pain and discomfort generally experienced due to an injury to this region of the body. The brace 10 can be readily removed and re-worn as desired by the patient or may be worn continually by the patient in accordance to the patient's needs.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

Orientational terms used in the specification and claims such as vertical, horizontal, top, bottom, upper and lower are to be interpreted as relational and are based on the premise that the component, item, article, apparatus, device or instrument will usually be considered in a particular orientation, typically with the brace uppermost.

It will be appreciated by those skilled in the art that many modifications and variations may be made to the methods of the invention described herein without departing from the spirit and scope of the invention.

The claims defining the invention are as follows:

1. A brace for treating an injury of the distal radio-ulna joint of an individual, comprising:
a semi-rigid support member having a concave surface configured to extend longitudinally over a distal radius of the individual adjacent the individual's distal radio-ulna joint;
at least one retaining arm configured to be wound about the individual's arm to engage with an outer surface of the support member;
wherein the at least one retaining arm is formed from a resilient material such that tightening of the retaining arm about the individual's arm applies pressure to the distal radio-ulnar joint to alleviate pain and discomfort experienced therein.

2. A brace according to claim 1, wherein the semi-rigid support member has a semi-cylindrical configuration that defines the concave surface so as to be fitted over and along the individual's radius, adjacent the distal radio-ulnar joint.

3. A brace according to claim 2, wherein the concave surface of the support member is coated with a resilient layer to act as a cushion between the support member and the individual's skin.

4. A brace according to claim 2, wherein support member is flexible about a longitudinal axis but rigid about a radial axis.

5. A brace according to any one of the preceding claims wherein the support member cups the distal radius when worn.

6. A brace according to claim 1, wherein the brace contains one retaining arm and the retaining arm is attachable by way of a hook and loop fastener to the outer surface of the support member.

7. A brace according to claim 6, wherein the retaining member is formed from a neoprene material.

8. A brace according to claim 2 wherein the support member is mounted over a pad member which is configured to be positioned between the support member and the individual's skin when in use.

9. A brace according to claim 8, wherein two retaining arms are provided to extend angularly from opposing ends of the pad member.

10. A brace according to claim 9, wherein the two retaining arms form a front retaining arm and a rear retaining arm which extend about a front region and a rear region of the support member to secure the brace in position on the arm of the individual.

11. A brace according to claim 10, wherein the two retaining arms are positioned so as to provide a right-handed version of the brace and a left-handed version of the brace for use on either a right or eft hand of the individual.
